SEQUENCE LISTING (3 sequences)

SEQ ID NO: 1 - DNA, 1482 bp, unidentified
  source 1..1482: mol_type = genomic DNA
cagggttatggctgacagctcgtacataacgggtcgaacagaactctggtattgattggtgcttgcatcatgatttacatttgagtgagtggcgaactggtgagtaacacgtgggaaacctgcccagaagcgggggataacacctggaaacagatgctaataccgcataacaacttggaccgcatggtccgagcttgaaagatggcttcggctatcacttttggatggtcccgcggcgtattagctagatggtggggtaacggctcaccatggcaatgatacgtagccgacctgagagggtaatcggccacattgggactgagacacggcccaaactcctacgggaggcagcagtagggaatcttccacaatggacgaaagtctgatggagcaacgccgcgtgagtgaagaagggtttcggctcgtaaaactctgttgttaaagaagaacatatctgagagtaactgttcaggtattgacggtatttaaccagaaagccacggctaactacgtgccagcagccgcggtaatacgtaggtggcaagcgttgtccggatttattgggcgtaaagcgagcgcaggcggttttttaagtctgatgtgaaagccttcggctcaaccgaagaagtgcatcggaaactgggaaacttgagtgcagaagaggacagtggaactccatgtgtagcggtgaaatgcgtagatatatggaagaacaccagtggcgaaggcggctgtctggtctgtaactgacgctgaggctcgaaagtatgggtagcaaacaggattagataccctggtagtccataccgtaaacgatgaatgctaagtgttggagggtttccgcccttcagtgctgcagctaacgcattaagcattccgcctggggagtacggccgcaaggctgaaactcaaaggaattgacgggggcccgcacaagcggtggagcatgtggtttaattcgaagctacgcgaagaaccttaccaggtcttgacatactatgcaaatctaagagattagacgttcccttcggggacatggatacaggtggtgcatggttgtcgtcagctcgtgtcgtgagatgttgggttaagtcccgcaacgagcgcaacccttattatcagttgccagcattaagttgggcactctggtgagactgccggtgacaaaccggaggaaggtggggatgacgtcaaatcatcatgccccttatgacctgggctacacacgtgctacaatggatggtacaacgagttgcgaactcgcgagagtaagctaatctcttaaagccattctcagttcggattgtaggctgcaactcgcctacatgaagtcggaatcgctagtaatcgcggatcagcatgccgcggtgaatacgttcccgggccttgtacacaccgcccgtcacaccatgagagtttgtaacacccaaagtcggtggggtaacctttgaggaaccagcctacctcatcgagtttcccccg

SEQ ID NO: 2 - DNA, 1483 bp, unidentified
  source 1..1483: mol_type = genomic DNA
agggggcggggtctatacatggcggggtcgaacgcactctcgtttagattgaaggagcttgctcctgattgataaacatttgagtgagtggcggacgggtgagtaacacgtgggtaacctgccctaaagtgggggataacatttggaaacagatgctaataccgcataaaacctaacaccgcatggtgtagggttgaaagatggtttcggctatcactttaggatggacccgcggtgcattagttagttggtgaggtaaaggctcaccaagaccgtgatgcatagccgacctgagagggtaatcggccacactgggactgagacacggcccagactcctacgggaggcagcagtagggaatcttccacaatggacgaaagtctgatggagcaacgccgcgtgagtgaagaaggttttcggatcgtaaaactctgttgttggagaagaatgtatctgatagtaactgatcaggtagtgacggtatccaaccagaaagccacggctaactacgtgccagcagccgcggtaatacgtaggtggcaagcgttgtccggatttattgggcgtaaagcgagcgcaggcggtttcttaagtctgatgtgaaagccttcggctcaaccgaagaagtgcatcggaaactgggaaacttgagtgcagaagaggacagtggaactccatgtgtagcggtgaaatgcgtagatatatggaagaacaccagtggcgaaggcggctgtctggtctgtaactgacgctgaggctcgaaagcatgggtagcaaacaggattagataccctggtagtccatgccgtaaacgatgagtgctaggtgttggagggtttccgcccttcagtgccgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagctttcccttcggggacaaagtgacaggtggtgcatggttgtcgtcagctcgtgtcgtgagatgttgggttaagtcccgcaacgagcgcaacccttattactagttgccagcatttagttgggcactctagtgagactgccggtgacaaaccggaggaaggtggggacgacgtcaaatcatcatgccccttatgacctgggctacacacgtgctacaatggatggtacaacgagttgcgagaccgcgaggtttagctaatctcttaaaaccattctcagttcggattgtaggctgcaactcgcctacatgaagccggaatcgctagtaatcgcggatcagcatgccgcggtgaatacgttcccgggccttgtacacaccgcccgtcacaccatgagagtttgtaacacccaaagccggtgaggtaacccttcgtggagctcagctgtctaaagttaaatcgggatg

SEQ ID NO: 3 - DNA, 1434 bp, unidentified
  source 1..1434: mol_type = genomic DNA
cggaacgcactctcgttagattgaagaagcttgcttctgattgataacatttgagtgagtggcggacgggtgagtaacacgtgggtaacctgccctaaagtgggggataacatttggaaacagatgctaataccgcataaaacctagcaccgcatggtgcaaggttgaaagatggtttcggctatcactttaggatggacccgcggtgcattagttagttggtgaggtaaaggctcaccaagaccgtgatgcatagccgacctgagagggtaatcggccacactgggactgagacacggcccagactcctacgggaggcagcagtagggaatcttccacaatggacgaaagtctgatggagcaacgccgcgtgagtgaagaaggttttcggatcgtaaaactctgttgttggagaagaacgtatttgatagtaactgatcaggtagtgacggtatccaaccagaaagccacggctaactacgtgccagcagccgcggtaatacgtaggtggcaagcgttgtccggatttattgggcgtaaagcgagcgcaggcggtttcttaagtctgatgtgaaagccttcggctcaaccgaagaagtgcatcggaaactgggaaacttgagtgcagaagaggacagtggaactccatgtgtagcggtgaaatgcgtagatatatggaagaacaccagtggcgaaggcggctgtctggtctgtaactgacgctgaggctcgaaagcatgggtagcaaacaggattagataccctggtagtccatgccgtaaacgatgagtgctaggtgttggagggtttccgcccttcagtgccgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagctttcccttcggggacaaagtgacaggtggtgcatggttgtcgtcagctcgtgtcgtgagatgttgggttaagtcccgcaacgagcgcaacccttattactagttgccagcatttagttgggcactctagtgagactgccggtgacaaaccggaggaaggtggggacgacgtcaaatcatcatgccccttatgacctgggctacacacgtgctacaatggatggtacaacgagtcgcgagaccgcgaggtttagctaatctcttaaaaccattctcagttcggattgtaggctgcaactcgcctacatgaagccggaatcgctagtaatcgcggatcagcatgccgcggtgaatacgttcccgggccttgtacacaccgcccgtcacaccatgagagtttgtaacacccaaagccggtgaggtaaccttcgggagccagccgtca

(19) United States
(12) Patent Application Publication
(10) Pub. No.: US 2024/0324636 A1
(43) Pub. Date: Oct. 3, 2024

(54) DRY POWDER FERMENTER AND APPLICATION THEREOF IN PREPARATION OF CURED MEAT PRODUCTS

(71) Applicant: Hefei University of Technology, Hefei City, Anhui Province (CN)

(72) Inventors: Baocai Xu, Hefei City, Anhui Province (CN); Xiaomin Li, Hefei City, Anhui Province (CN); Liu Yang, Hefei City, Anhui Province (CN); Jieying Deng, Hefei City, Anhui Province (CN); Yongsheng Ni, Hefei City, Anhui Province (CN)

(21) Appl. No.: 18/420,136

(22) Filed: Jan. 23, 2024

(30) Foreign Application Priority Data
Mar. 29, 2023 (CN) 202310324807.X

(51) Int. Cl. A23L 13/40

(52) U.S. Cl. CPC A23L 13/46; A23L 13/428

(57) ABSTRACT

A dry powder fermenter and an application thereof in preparation of cured meat products. The dry powder fermenter includes Lactiplantibacillus plantarum YR07, Latilactobacillus sakei L. 48, and Lactobacillus curvatus D2. The dry powder fermenter prepared by the present disclosure can be applied in indigenous fermented meat products, which may significantly shorten the fermentation time of the meat products and improve the flavor of the fermented meat products, with the fastest fermentation time being 4 days.

### CROSS REFERENCE

The present disclosure claims priority of Chinese Patent Application No. 202310324807. X, filed on Mar. 29, 2023, the entire contents of which are hereby incorporated by reference in their entirety.

### INCORPORATION-BY-REFERENCE OF MATERIAL in an XML FILE

The contents of the electronic sequence listing (File Name:15.xml; Size: 7858 bytes; and Date of Creation: Jan. 22, 2024) is hereby incorporated herein by reference in its entirety.

### TECHNICAL FIELD

The present disclosure relates to field of microbial fermentation technologies, especially relates to a dry powder fermenter and an application thereof in preparation of cured meat products.

### BACKGROUND

Fermenters are cultures of bacteria and other microorganisms used in the production and preparation of fermented products, which are capable of standardizing production characteristics of the products, competing with and inhibiting the activity of stray bacteria in the products, shortening the fermentation time, and improving the sensory characteristics of the products. In addition, some of the fermenters are capable of rapidly lowering the pH of the products, inhibiting the growth of microorganisms with amino acid decarboxylase ability, and thereby inhibiting the production of biogenic amines in the products. The preparation process of dry powder fermenter includes strain multiplication and culture, collection and concentration, and drying.

Lactic acid bacteria, an important component of the fermenter, play a dual role in fermented products: 1) to inhibit or control the growth of food pathogenic microorganisms or food spoilage microorganisms; and 2) to prolong the shelf-life of the product while maintaining its sensory characteristics, including color, flavor, texture, and nutritional value. Lactic acid bacteria constituting fermenters inhibit or reduce the growth of spoilage/pathogenic flora mainly through mechanisms such as the production of certain metabolites or competitive exclusion. Lactic acid bacteria are applied in fermented foods mainly to ferment carbohydrates in the products into lactic acid, which rapidly acidifies the food matrix and thus serves to improve the shelf life and biosecurity of the products. Also, most of the lactic acid bacteria have a large contribution to the flavor formation of fermented foods.

In addition, Lactic acid bacteria are important probiotic flora, where several strains are present in the small intestinal environment, which are resistant to the low pH extremes of the stomach, the presence of bile salts in the intestinal phase, and other natural growth inhibitors, and are able to interact with other microorganisms to adhere and colonize the gastrointestinal tract, to compete for nutrients with harmful bacteria, and to secrete relevant antimicrobial substances that inhibit the growth of harmful bacteria, thereby improving the intestinal flora and environment and thus exerting its beneficial ability to the host. In addition, lactic acid bacteria can reduce the level of intestinal diseases (e.g., inflammatory bowel syndrome, improving blood cholesterol levels), enhance lactose digestion, stimulate the immune system, and prevent and treat diarrhea.

The growth and cultivation of lactic acid bacteria are generally carried out using liquid media. However, liquid formulations are large in volume and difficult to transport, especially when transported at room temperature, where the bacterial viability decreases rapidly and it is difficult to achieve the original fermentation capacity. Therefore, minimizing the moisture content and maintaining the viability of the original strain are the commonly used techniques for the preparation of fermenters. Among the various drying techniques, freeze drying is the main method for the preparation of dry powder fermenters.

Freeze drying starts from freezing, followed by sublimation and desorption of water in the product, which is widely used for preserving lactic acid bacteria because of its long shelf life and low damage to cells. However, even with this method, the cells are subjected to a variety of stresses that cause them to lose viability and functional activity during the freeze-drying process. The main forms of damage to cells by freeze drying include mechanical damage, solute damage, cell membrane damage, metabolic damage, and DNA damage. Currently, the commonly used method to improve the survival rate of bacterial after freeze-drying is to add protective agents. However, for the production of industrial-scale microorganisms, the use of the protective agents alone does not achieve a satisfactory freeze-drying survival rate. Therefore, the method of changing the growth conditions, adding multiplication factors and fatty acids, and changing the nature of the cell membrane of the bacterial cells, in combination with the protective agent, can better improve the survival rate of the bacterium and the fermentation viability of the strain during the freeze-drying process, thereby improving the stability and sensory characteristics of the indigenous fermented meat products.

### SUMMARY OF THE DISCLOSURE

It is the object of the present disclosure to provide a dry powder fermenter and an application thereof in preparation of cured meat products.

In order to achieve the above and other related purposes, the present disclosure provides the following technical solutions: a dry powder fermenter, comprising Lactiplantibacillus plantarum YR07, Latilactobacillus sakei L. 48, and Lactobacillus curvatus D2; wherein the Lactiplantibacillus plantarum YR07 has been conserved in China Center for Type Culture Collection (CCTCC), with a conservation date of Aug. 18, 2022, and a strain conservation number of CCTCC NO:M 20221303; the Latilactobacillus sakei L. 48 has been conserved in the CCTCC, with a conservation date of Aug. 18, 2022, and a strain conservation number of CCTCC NO: M 20221306; the Lactobacillus curvatus D2 has been conserved in the CCTCC, with a conservation date of Feb. 21, 2023, and a strain conservation number of CCTCC NO: M 2023162.

In some embodiments, the dry powder fermenter is prepared by:
Step 1: inoculating the Lactiplantibacillus plantarum YR07, the Latilactobacillus sakei L. 48, and the Lactobacillus curvatus D2 into an MRS broth medium, respectively, and incubating at 35-39° C. at a rotational speed of 120-160 rpm under shaking; carrying out passaging culture for 3 times at an inoculum of 1.8-2.2%; adding strains to an MRS broth medium containing a multiplication factor at an inoculum of 1.8-2.2%, and culturing at 35-39° C. with a rotational speed of 120-160 rpm under shaking;
Step 2: when an optical density of a culture resulted in step 1 reaches 0.8-1.2, centrifuging and collecting the culture to obtain Lactiplantibacillus plantarum YR07 bacterial cells, Latilactobacillus sakei L. 48 bacterial cells, and Lactobacillus curvatus D2 bacterial cells; and
Step 3: suspending the Lactiplantibacillus plantarum YR07 bacterial cells, Latilactobacillus sakei L. 48 bacterial cells, and Lactobacillus curvatus D2 bacterial cells in sterile saline, mixing with a skimmed milk solution containing a freeze-drying protective agent and a fatty acid, and performing freeze-drying to obtain the dry powder ferment.

In some embodiments, the multiplication factor is at least one of an amino acid, a buffer salt, and vegetable juice.

In some embodiments, the amino acid is at least one of aspartic acid, phenylalanine, histidine, valine, and lysine, at a concentration of 0.002 wt %-0.008 wt %.

In some embodiments, the buffer salt is: disodium hydrogen phosphate and sodium dihydrogen phosphate, disodium hydrogen phosphate and citric acid, potassium dihydrogen phosphate and sodium hydroxide, sodium acetate, potassium hydrogen phosphate and sodium citrate, or disodium hydrogen phosphate and potassium dihydrogen phosphate.

In some embodiments, the vegetable juice is at least one of tomato juice, carrot juice, and corn juice.

In some embodiments, in the step 1, a culture condition in each time of the passaging culture is the same: being cultured for 18-20 h at 35-39° C. at a rotational speed of 120-160 rpm; each liter of the MRS medium contains peptone 10.0 g, beef meal 5.0 g, yeast powder 4.0 g, glucose 20.0g\Tween 80 1 mL\K₂HPO₄·7H₂O 2.0 g, triammonium citrate 2.0 g, magnesium sulfate 0.2 g; manganese sulfate, 0.05 g; pH 6.2±0.2;
in the step 2, a centrifugal force is 6000-10000 g; a centrifugal time is 10-30 min, and a temperature at centrifugation is 2-5° C.

In some embodiments, the freeze-drying protective agent is at least one of mannitol, sucrose, peptone, and alginate, and the fatty acid is at least one of oleic acid, linoleic acid, palmitic acid, and palmitoleic acid; a concentration of the fatty acid in the skimmed milk solution containing the freeze-drying protective agent and the fatty acid is 0.01 v/v %-0.05 v/v %; a concentration of the skimmed milk solution is 8 wt %-12 wt %; a volume ratio of a suspension of the bacterial cells to the skimmed milk solution containing the freeze-drying protective agent and the fatty acid is 2:1-1:2; and a duration of the freeze-drying is 24-30 h.

In order to achieve the above and other related purposes, the present disclosure provides the following technical solutions: an application of the dry powder fermenter in a preparation of cured meat products.

In some embodiments, the cured meat product is a dry-cured duck, dry-cured chicken, traditional Chinese bacon (Dao-ban-xiang), or ham product.

As a result of the use of the above technical solutions, the present disclosure has the following advantages over the related art.
1, the present disclosure improves the survival rate and fermentation vigor of the bacterial strain by using the method of combining the method of changing the growth conditions (addition of multiplication factor) with the method of changing the composition of freeze-drying protective agent (addition of fatty acids) for the preparation of the dry powder fermenter.
2, a simple multiplication factor addition is applied for obtaining a high cell concentration of the bacterium, and forming a protective layer of bacterial gelatin clusters, thereby reducing the exposure of the bacterial cells in the environment, increasing its resistance to the unfavorable conditions of freeze-drying, and improving the survival rate of the dry powder fermenter.
3, the method of adding fatty acids is applied to change the composition of fatty acids of the cell membrane of the bacterium, thereby improving the membrane fluidity of the cells, improving the resistance to freeze-drying of the bacterium strain, and improving the viability and fermentation vigor of the bacterium strain.
4, the preparation method of the present disclosure is simple, and the dry powder fermenter prepared by the method of the present disclosure can be applied to indigenous fermented meat products, which can significantly shorten the fermentation time of the meat products and improve the flavor of the fermented meat products, and the fastest fermentation time is 4 days.

### BRIEF DESCRIPTION OF THE DRAWINGS

FIG. 1 shows the freeze-drying survival rate of isolated lactic acid bacteria strains in the embodiments, without strain proliferation multiplication and culture and addition of fatty acids, and after freeze-drying treatment. It can be seen from FIG. 1 that different strains have different freeze-drying survival rates and the strains have lower freeze-drying survival rates.

FIG. 2 shows the effect of adding different kinds of amino acids on the freeze-drying survival rate of Lactiplantibacillus plantarum in the embodiments. It can be seen from FIG. 2 that different amino acids affect the freeze-drying survival rate of the strains, and aspartic acid is able to increase the freeze-drying survival rate of the strains.

FIG. 3 shows the effect of different concentrations of phosphates on the freeze-drying survival of the strains in the embodiments. It can be seen from FIG. 3 that the freeze-drying survival rate of the strains is first increased and then decreased with the increase of phosphate concentration, and at a concentration of 0.10 mol/L, the freeze-drying survival rate is higher.

FIG. 4 shows the effect of different fatty acid species on the freeze-drying survival rate of the strains in the embodiments. It can be seen from FIG. 4 that oleic acid is able to increase the freeze-drying survival rate of the strains, and the chain length of the fatty acid also affects the freeze-drying survival rate of the strains.

FIG. 5 shows the effect of different adding multiplication factors and fatty acids on the number of viable bacteria in the embodiments. It can be seen from FIG. 5 that the number of viable bacteria is higher after treatment with aspartic acid, phosphate buffer, and oleic acid.

FIG. 6 shows the effect of different adding multiplication factors and fatty acids on the freeze-drying survival of the strains in the embodiments. It can be seen from FIG. 6 that treatment with aspartic acid, phosphate buffer, and oleic acid substantially increases the freeze-drying survival rate of the strains.

FIG. 7 shows the effect of the embodiments on the flavor of fermented meat products compared to the dry powder fermenter prepared by conventional addition of only skim milk powder freeze-drying protective agent, wherein C-1, C-2, and C-3 are fermented samples of the dry powder fermenter prepared by addition of only skim milk powder, and T-1, T-2, and T-3 are fermented meat samples of the dry powder fermenter prepared in the embodiments. As can be seen from the FIG. 7, the dry powder fermenter prepared using the method of the present disclosure has high fermentation activity and is capable of enhancing the flavor of the fermented meat product.

### DETAILED DESCRIPTION

The following specific embodiments illustrate the implementation of the present disclosure, such that those skilled in the art can easily understand other advantages and effects of the present disclosure from the contents disclosed herein.

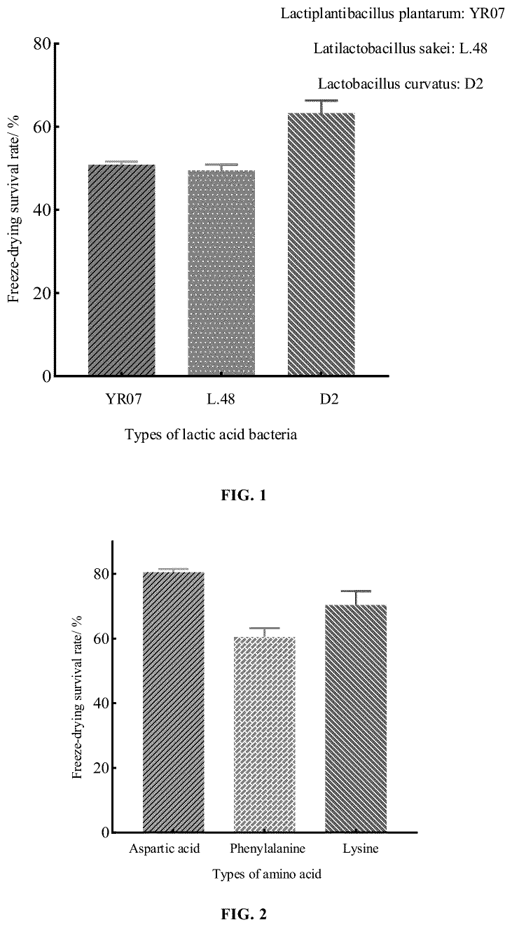

Referring to FIGS. 1-7, it is noted that the structures, proportions, sizes, etc. shown in the accompanying drawings in this specification are only intended to be in conjunction with the contents of the specification for the benefit of those skilled in the art, and are not intended to limit the conditions under which the present disclosure may be practiced, are not intended for technical purposes, and do not imply any modification of the structure, change in proportionality, or adjustment of the size of the present disclosure. The following embodiments are provided for a better understanding of the present disclosure and are not intended to limit the present disclosure. The experimental methods in the following embodiments are conventional if not otherwise specified. The experimental materials used in the following embodiments are purchased from conventional biochemical reagent stores, if not otherwise specified.

### Preservation of Biological Materials

The Lactiplantibacillus plantarum YR07, has been conserved in the China Center for Type Culture Collection (CCTCC), with a conservation date of Aug. 18, 2022, and a strain conservation number of CCTCC NO:M 20221303; the Latilactobacillus sakei L. 48, has been conserved in the CCTCC, with a conservation date of Aug. 18, 2022, and a strain conservation number of CCTCC NO: M 20221306; the Lactobacillus curvatus D2, has been conserved in the CCTCC, with a conservation date of Feb. 21, 2023, and a strain conservation number of CCTCC NO: M 2023162. The conservation address: CCTCC (Wuhan, China).

The reagents or materials described in the following embodiments are commercially available if not stated.

### Embodiment 1: A Dry Powder Fermenter and its Application in the Preparation of Cured Meat Products

### 1: Multiplication Culture of Lactic Acid Bacteria

Latilactobacillus sake is mainly isolated from dry-cured ducks and stored in a refrigerator at −80° C. using a magnetic bead preservation method. The activation of the strain is carried out by picking a single magnetic bead in MRS broth culture in a constant temperature incubator at 37° C. with shaking at 150 rpm for 24 h. Subsequently, the strain is passaged at 2% inoculum for 3 times and incubated at 37° C. for 18 h at 150 rpm to ensure the viability of the strain.

Subsequently, the strain is added into a medium containing 0.002 wt % aspartic acid and 0.14 mol/L mixed buffer salt of disodium hydrogen phosphate and citric acid at 2% inoculum, and incubated at 37° C. in a constant temperature incubator under the condition of 150 rpm with shaking.

### 2: Collection and Concentration of Bacterial Cells

When the optical density (OD600) of the culture reaches 1, an appropriate amount of bacterial culture is taken into a sterile 50 mL centrifuge tube, centrifuged at 8000 g for 15 min at 4° C. to collect the bacterial cells, washed with sterile saline, and centrifuged again under the same conditions to concentrate and collect the bacterial cells.

### 3: Preparation of High Vigor Bacterial Powder Fermenter

0.02 v/v % oleic acid is added into 10 wt % skimmed milk containing mannitol, and the above collected bacterial cells are suspended in sterile saline and mixed with the skimmed milk solution containing freeze-drying protective agent and fatty acid with a volume ratio of 2:1, and then subsequently dispensed into syringes per 5 mL. The freezing program is set as follows: the samples are frozen at −80° C. for 3 h, the cold trap temperature is −60° C., the vacuum is less than 10 Kpa Hg, and the samples are freeze-dried for 24 h.

### 4. Preparation of Fermented Meat Products

Duck meat of suitable weight and size is taken, and 2 wt % salt is added to coat it evenly and left to stand at 4° C. for 24 h. The duck meat is immersed in cooled brine containing 0.02% vanilla powder, 1 wt % onion, 1 wt % ginger, 0.02 wt % nutmeg, 0.02 wt % allspice, and 0.05 wt % peppercorns, and after the immersion is completed, the duck meat is taken out and drained for treatment, followed by adding dry powder fermenter (with an inoculum of 1×10⁷ CFU/g), and fermentation is carried out for 4 days in a fermentation room at 10-15° C. and 65% relative humidity.

### Embodiment 2: A Dry Powder Fermenter and its Application in the Preparation of Cured Meat Products

### 1: Multiplication Culture of Lactic Acid Bacteria

Lactiplantibacillus plantarum is mainly isolated from fermented ham and stored in a refrigerator at −80° C. using a magnetic bead preservation method. The activation of the strain is carried out by picking a single magnetic bead in MRS broth culture in a constant temperature incubator at 37° C. with shaking at 150 rpm for 24 h. Subsequently, the strain is passaged at 2% inoculum for 3 times and incubated at 37° C. for 18 h at 150 rpm to ensure the viability of the strain.

Subsequently, the strain is added into the medium containing 0.004 wt % phenylalanine and 0.10 mol/L mixed buffer salt of disodium hydrogen phosphate and sodium dihydrogen phosphate at 2% inoculum, and incubated at 37° C. in a constant temperature incubator under the condition of 150 rpm with shaking.

### 2: Collection and Concentration of Bacterial Cells

When the optical density (OD600) of the culture reaches 1, an appropriate amount of bacterial culture is taken into a sterile 50 mL centrifuge tube, centrifuged at 6000 g for 20 min at 4° C. to collect the bacterial cells, washed with sterile saline, and centrifuged again under the same conditions to concentrate and collect the bacterial cells.

### 3: Preparation of High Vigor Bacterial Powder Fermenter

0.03 v/v % oleic acid is added into 8 wt % skimmed milk containing alginate, and the above collected bacterial cells are suspended in sterile saline and mixed with the skimmed milk solution containing freeze-drying protective agent and fatty acids with a volume ratio of 1:1, and then subsequently dispensed into syringes per 5 mL. The freezing program is set as follows: the samples are frozen at −80° C. for 3 h, the cold trap temperature is −60° C., the vacuum is less than 10 Kpa Hg, and the samples are freeze-dried for 28 h.

### 4. Preparation of Fermented Meat Products

Chicken meat of suitable weight and size is taken, and 2 wt % salt is added to coat it evenly and left to stand at 4° C. for 24 h. The chicken meat is immersed in cooled brine containing 0.02% vanilla powder, 1 wt % onion, 1 wt % ginger, 0.02 wt % nutmeg, 0.02 wt % allspice, and 0.05 wt % peppercorns, and after the immersion is completed, the chicken meat is taken out and drained for treatment, followed by adding dry powder fermenter (with an inoculum of 1×10⁷ CFU/g), and fermentation is carried out for 4 days in a fermentation room at 10-15° C. and 65% relative humidity.

### Embodiment 3: A Dry Powder Fermenter and its Application in the Preparation of Cured Meat Products

### 1: Multiplication Culture of Lactic Acid Bacteria

Lactobacillus curvatus is mainly isolated from fermented ham and stored in a refrigerator at −80° C. using a magnetic bead preservation method. The activation of the strain is carried out by picking a single magnetic bead in MRS broth culture in a constant temperature incubator at 37° C. with shaking at 150 rpm for 24 h. Subsequently, the strain is passaged at 2% inoculum for 3 times and incubated at 37° C. for 18 h at 150 rpm to ensure the viability of the strain.

Subsequently, the strain is added into the medium containing 0.008 wt % histidine and 0.08 mol/L mixed buffer salt of disodium hydrogen phosphate and potassium dihydrogen phosphate at 2% inoculum, and incubated at 37° C. in a constant temperature incubator under the condition of 150 rpm with shaking.

### 2: Collection and Concentration of Bacterial Cells

When the optical density (OD600) of the culture reaches 1, an appropriate amount of bacterial culture is taken into a sterile 50 mL centrifuge tube, centrifuged at 10,000 g for 10 min at 4° C. to collect the bacterial cells, washed with sterile saline, and centrifuged again under the same conditions to concentrate and collect the bacterial cells.

### 3: Preparation of High Vigor Bacterial Powder Fermenter

0.04 v/v % palmitoleic acid is added into 12 wt % skimmed milk containing peptone, and the above collected bacterial cells are suspended in sterile saline and mixed with the skimmed milk solution containing freeze-drying protective agent and fatty acids with a volume ratio 1:2, and then subsequently dispensed in syringes per 5 mL. The freezing program is set as follows: the samples are frozen at −80° C. for 3 h, the cold trap temperature is −60° C., the vacuum is less than 10 Kpa Hg, and the samples are freeze dried for 30 h.

### 4. Preparation of Fermented Meat Products

Pork meat of suitable weight and size is taken, and 2 wt % salt is added to coat it evenly and left to stand at 4° C. for 24 h. The pork meat is immersed in cooled brine containing 0.02% vanilla powder, 1 wt % onion, 1 wt % ginger, 0.02 wt % nutmeg, 0.02 wt % allspice, and 0.05 wt % peppercorns, and after the immersion is completed, the pork meat is taken out and drained for treatment, followed by adding dry powder fermenter (with an inoculum of 1×10⁷ CFU/g), and fermentation is carried out for 4 days in a fermentation room at 10-15° C. and 65% relative humidity.

### Embodiment 4: A Dry Powder Fermenter and its Application in the Preparation of Cured Meat Products

A dry powder fermenter, containing Lactiplantibacillus plantarum YR07 obtained by isolation from dry-cured duck, under the conservation number of CCTCC NO: M 2021303; the Latilactobacillus sakei L. 48, under the conservation number of CCTCC NO: M 20221306; the Lactobacillus curvatus D2, under the conservation number of CCTCC NO: M 2023162; the conservation address is CCTCC (Wuhan, China); the preparation of the highly active dry powder fermenter is processed according to the following steps: 1) obtaining a high density of bacterial cells under lactic acid bacteria growth conditions (addition of multiplication factors: amino acids; buffer salts; growth-promoting factors), and centrifuging to collect the bacterial cells, and 2) adding into a skimmed milk solution containing variable freeze-drying protective agent (with fatty acids), and vacuum freeze-drying treatment to prepare a small-sized and easy-to-transport dry powder fermenter for indigenous fermentation of meat products; the prepared dry powder fermenter can be used in the production of indigenous fermented meat such as dry-cured duck, fermented sausage, air-dried ham, etc.

The method of preparation includes the following steps:

Step 1: Isolation of Lactic Acid Bacteria from Indigenous Fermented Meat Products

Indigenous products are purchased from local workshops, aseptic homogenization bags are applied for sampling, the sample is mixed with sterile saline for treatment, and dilution is performed with a series of gradient; a suitable gradient of the dilution solution is selected and coated on a MRS plate containing CaCO₃—, and placed in a 37° C. incubator incubation for 48 h; from the bacterial number of 30-300 on the plate, colonies with different colors and sizes are picked for delimitation purification treatment; single colony obtained is subjected to a series of property measurements, including safety assessment, fermentation characteristics, protease and lipase production capacity; a strain with good safety, fermentation characteristics, protease and lipase production is selected for DNA extraction and amplification by PCR (primers 27F and 1492R), and sent to Shanghai Bio-engineering Biological Company for sequencing. In addition, the obtained lactic acid bacteria are sent to CCTCC for conservation.

### Step 2: Preparation of Concentrated High-Activity Dry Powder Fermenter for Indigenous Fermented Meat Products

### 1) Multiplication Culture of Lactic Acid Bacteria

The lactic acid bacteria strain is inoculated in MRS broth culture in a constant temperature incubator at 37° C. with shaking at 150 rpm for 24 h. Subsequently, the strain is passaged at 2% inoculum for 3 times and incubated at 37° C. for 18 h at 150 rpm to ensure the viability of the strain.

Subsequently, the strain is added to an MRS broth medium containing different multiplication factors (amino acids, buffer salts, growth-promoting factors) at 2% inoculum, and incubated at 37° C. in a constant temperature incubator with shaking at 150 rpm.

### 2) Collection and Concentration of Bacterial Cells

The lactic acid bacteria are multiplicated and cultured under the conditions of step 1, and when the optical density (OD600) of the culture reaches 1, an appropriate amount of bacterial culture is taken into a sterile 50 mL centrifuge tube, and the bacterial cells are concentrated and collected by changing the size of the centrifugal force and the length of the centrifugal time at 4° C.

### 3) Preparation of High Vigor Bacterial Powder Fermenter

A skimmed milk solution containing common freeze-drying protective agents is configured, the collected bacterial cells are suspended in sterile saline and mixed with the skimmed milk solution containing the freeze-drying protective agents and fatty acids with a volumetric ratio, and then subsequently dispensed in syringes per 5 mL. The freezing program is set as follows: the samples are frozen at −80° C. for 3 h, with a cold trap temperature of −60° C. and a vacuum of less than 10 Kpa Hg.

### Step 3: Preparation of Fermented Meat Products

Cleaned and drained duck meat is taken, and 2 wt % salt is added to coat it evenly and left to stand at 4° C. for 24 h. The duck is immersed in cooled brine containing 0.02% vanilla powder, 1 wt % onion, 1 wt % ginger, 0.02 wt % nutmeg, 0.02 wt % allspice, and 0.05 wt % peppercorns, and after the immersion is completed, the duck meat is taken out and drained for treatment, followed by adding dry powder fermenter (with an inoculum of 1×10⁷ CFU/g), and fermentation is carried out for 4 days in a fermentation room at 10-15° C. and 65% relative humidity.

In the step 2, the multiplication factor may be an amino acid such as: aspartic acid, phenylalanine, histidine, valine, and lysine, at a concentration of 0.002 wt %-0.008 wt %.

The multiplication factor may be different buffer salts such as: disodium hydrogen phosphate and sodium dihydrogen phosphate; disodium hydrogen phosphate and citric acid; potassium dihydrogen phosphate and sodium hydroxide; sodium acetate, potassium hydrogen phosphate and sodium citrate; and disodium hydrogen phosphate and potassium dihydrogen phosphate; at a concentration of 0.8 mol/L-0.14 mol/L.

The multiplication factor may be a growth promoting factor such as tomato juice, carrot juice and corn juice. In the embodiment, the specific multiplication factor is carrot juice.

The centrifugal force of the bacterial cells is 8000 g; the centrifugal time is 20 min, and the centrifugal temperature is 4° C.

The type of the freeze-drying protective agent described is mannitol, sucrose, peptone, alginate, and the like. In the embodiment, the freeze-drying protective agent is specifically sucrose.

The kind of the fatty acid is: oleic acid, linoleic acid, palmitic acid, palmitoleic acid, which is specifically linoleic acid in the present embodiment; the concentration of the fatty acid is 0.01 v/v %-0.05 v/v %, which is specifically 0.03 v/v % in the present embodiment; the concentration of the skim milk solution is 8 wt %-12 wt %, which is specifically 10 wt % in the present embodiment; the volume ratio of the suspended bacterial solution to the skim milk solution containing freeze-drying protective agent and fatty acid is 2:1-1:2, which is specifically 1:1 in the present embodiment; the freeze-drying time is 24-30 h, which is specifically 18 h in the present embodiment.

In the dry powder fermenter, the survival rate of the fermentation bacterial strain is 81.5%-92%, and the fermentation activity is strong.

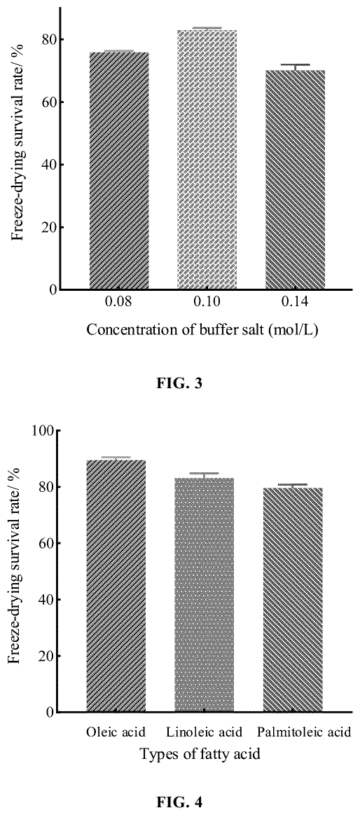

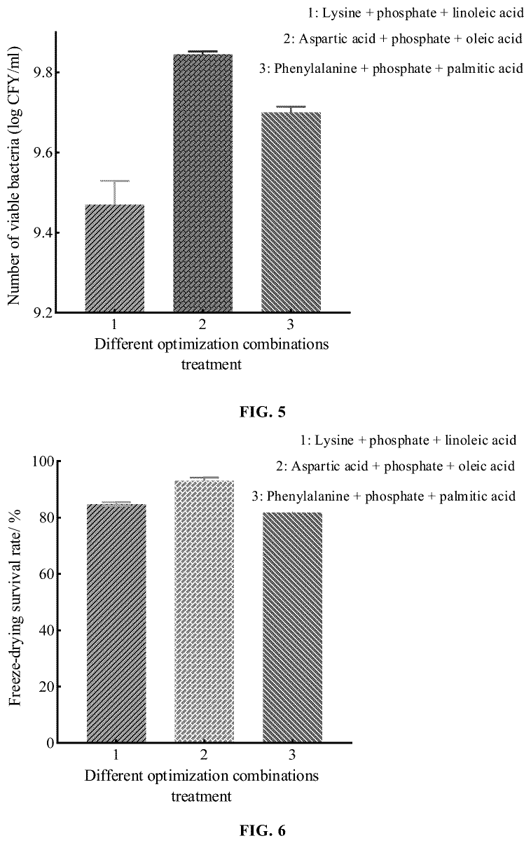

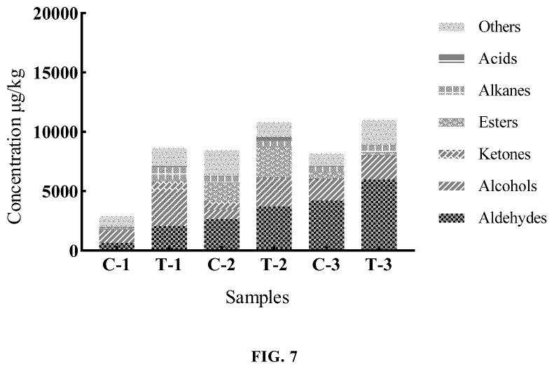

It can be seen from FIG. 1 that different strains have different freeze-drying survival rates and the strains have lower freeze-drying survival rates. It can be seen from FIG. 2 that different amino acids affect the freeze-drying survival rate of the strains, and aspartic acid is able to increase the freeze-drying survival rate of the strains. It can be seen from FIG. 3 that the freeze-drying survival rate of the strains is first increased and then decreased with the increase of phosphate concentration, and at a concentration of 0.10 mol/L, the freeze-drying survival rate is higher. It can be seen from FIG. 4 that oleic acid is able to increase the freeze-drying survival rate of the strains, and the chain length of the fatty acid also affects the freeze-drying survival rate of the strains. It can be seen from FIG. 5 that the number of viable bacteria is higher after treatment with aspartic acid, phosphate buffer, and oleic acid. It can be seen from FIG. 6 that treatment with aspartic acid, phosphate buffer, and oleic acid substantially increases the freeze-drying survival rate of the strains. FIG. 7 shows the effect of the embodiments on the flavor of fermented meat products compared to the dry powder fermenter prepared by conventional addition of only skim milk powder freeze-drying protective agent, wherein C-1, C-2, and C-3 are fermented samples of the dry powder fermenter prepared by addition of only skim milk powder, and T-1, T-2, and T-3 are fermented meat samples of the dry powder fermenter prepared in the embodiments. As can be seen from the FIG. 7, the dry powder fermenter prepared using the method of the present disclosure has high fermentation activity and is capable of enhancing the flavor of the fermented meat product.

### Embodiment 5: A Dry Powder Fermenter and its Application in the Preparation of Cured Meat Products

A dry powder fermenter, including Lactiplantibacillus plantarum YR07, Latilactobacillus sakei L. 48, and Lactobacillus curvatus D2; the Lactiplantibacillus plantarum YR07 has been conserved in the China Center for Type Culture Collection (CCTCC), with a conservation date of Aug. 18, 2022, and a strain conservation number of CCTCC NO:M 20221303; the Latilactobacillus sakei L. 48 has been conserved in the CCTCC, with a conservation date of Aug. 18, 2022, and a strain conservation number of CCTCC NO: M 20221306; the Lactobacillus curvatus D2 has been conserved in the CCTCC, with a conservation date of Feb. 21, 2023, and a strain conservation number of CCTCC NO: M 2023162.

The method of preparation includes the following steps:
Step 1: inoculating Lactiplantibacillus plantarum YR07, Latilactobacillus sakei L. 48, and Lactobacillus curvatus D2 into MRS broth medium, respectively, and incubating at 35° C. with shaking at 120 rpm; subsequently, passaging culture is carried out for 3 times at an inoculum of 1.8%; subsequently, adding the strain to an MRS broth medium containing multiplication factors at an inoculum of 1.8%, and cultured at 35° C. with a rotational speed of 120 rpm under shaking;
Step 2: when the optical density of the culture of step 1 reaches 0.8, centrifuging and collecting the bacterial culture to obtain Lactiplantibacillus plantarum YR07 bacterial cells, Latilactobacillus sakei L. 48 bacterial cells, and Lactobacillus curvatus D2 bacterial cells;
Step 3: suspending the Lactiplantibacillus plantarum YR07 bacterial cells, Latilactobacillus sakei L. 48 bacterial cells, and Lactobacillus curvatus D2 bacterial cells in sterile saline, mixing with a skimmed milk solution containing freeze-drying protective agent and fatty acid, and then freeze-dried to obtain a dry powder ferment.

In some embodiments, the multiplication factor is an amino acid.

In some embodiments, the amino acid is a mixture consisting of aspartic acid, phenylalanine in a mass ratio of 1:1 at a concentration of 0.006 wt %.

In some embodiments, in the step 1, the culture conditions are the same in the passaging culture, each of which is cultured for 18 h at 35° C. rotating at 120 rpm; each liter of the MRS medium contains peptone 10.0 g, beef meal 5.0 g, yeast powder 4.0 g, glucose 20.0 g\Tween 80 1 mL\K₂HPO₄·7H₂O 2.0 g, triammonium citrate 2.0 g, magnesium sulfate 0.2 g; manganese sulfate, 0.05 g; pH 6.

In the step 2, the centrifugal force is 6000 g; the centrifugal time is 10 min, and the temperature at centrifugation is 2° C.

In some embodiments, the freeze-drying protective agent is a mixture of mannitol and sucrose in a mass ratio of 1:1, and the fatty acid is a mixture of oleic acid and linoleic acid in a volumetric ratio of 1:1; the concentration of the fatty acid in the skimmed milk solution containing the freeze-drying protective agent and the fatty acid is 0.01 v/v %; the concentration of the skimmed milk solution is 8 wt %; a volume ratio of the bacterial cell suspension to the skimmed milk solution containing the freeze-drying protective agent and the fatty acid is 2:1; and the freeze-drying time is 24 h.

The dry powder fermenter can be applied in the preparation of ham.

### Embodiment 6: A Dry Powder Fermenter and its Application in the Preparation of Cured Meat Products

A dry powder fermenter, including Lactiplantibacillus plantarum YR07, Latilactobacillus sakei L. 48, and Lactobacillus curvatus D2; the Lactiplantibacillus plantarum YR07 has been conserved in the China Center for Type Culture Collection (CCTCC), with a conservation date of Aug. 18, 2022, and a strain conservation number of CCTCC NO:M 20221303; the Latilactobacillus sakei L. 48 has been conserved in the CCTCC, with a conservation date of Aug. 18, 2022, and a strain conservation number of CCTCC NO: M 20221306; the Lactobacillus curvatus D2 has been conserved in the CCTCC, with a conservation date of Feb. 21, 2023, and a strain conservation number of CCTCC NO: M 2023162.

The method of preparation includes the following steps:
Step 1: inoculating Lactiplantibacillus plantarum YR07, Latilactobacillus sakei L. 48, and Lactobacillus curvatus D2 into MRS broth medium, respectively, and incubating at 39° C. with shaking at 160 rpm; subsequently, passaging culture is carried out for 3 times at an inoculum of 2.2%; subsequently, adding the strain to an MRS broth medium containing multiplication factors at an inoculum of 2.2%, and cultured at 39° C. with a rotational speed of 160 rpm under shaking;
Step 2: when the optical density of the culture of step 1 reaches 1.2, centrifuging and collecting the bacterial culture to obtain Lactiplantibacillus plantarum YR07 bacterial cells, Latilactobacillus sakei L. 48 bacterial cells, and Lactobacillus curvatus D2 bacterial cells;
Step 3: suspending the Lactiplantibacillus plantarum YR07 bacterial cells, Latilactobacillus sakei L. 48 bacterial cells, and Lactobacillus curvatus D2 bacterial cells in sterile saline, mixing with a skimmed milk solution containing freeze-drying protective agent and fatty acid, and then freeze-dried to obtain a dry powder ferment.

In some embodiments, the multiplication factor is buffered salt.

In some embodiments, the buffer salt is: disodium hydrogen phosphate and sodium dihydrogen phosphate.

In some embodiments, in the step 1, the culture conditions are the same in the passaging culture, each of which is cultured for 20 h at 39° C. rotating at 160 rpm; each liter of the MRS medium contains peptone 10.0 g, beef meal 5.0 g, yeast powder 4.0 g, glucose 20.0 g\Tween 80 1 mL\K₂HPO₄·7H₂O 2.0 g, triammonium citrate 2.0 g, magnesium sulfate 0.2 g; manganese sulfate, 0.05 g; pH 6.4.

In the step 2, the centrifugal force is 10000 g; the centrifugal time is 30 min, and the temperature at centrifugation is 5° C.

In some embodiments, the freeze-drying protective agent is peptone, the fatty acid is palmitic acid; the concentration of the fatty acid in the skimmed milk solution containing the freeze-drying protective agent is 0.05 v/v %; the concentration of the skimmed milk solution is 12 wt %; a volume ratio of the bacterial cell suspension to the skimmed milk containing the freeze-drying protective agent and the fatty acid is 1:2; and the freeze-drying time is 30 h.

The dry powder fermenter can be applied in the preparation of traditional Chinese bacon (Dao-ban-xiang).

The dry powder fermenter prepared in the present disclosure can be applied in the production of indigenous fermented meat such as dry-cured duck, dry-cured chicken, and traditional Chinese bacon (Dao-ban-xiang), etc. The survival rate of the fermentation strain in the present disclosure is 81.5%-92%, which has excellent fermentation activity. Further, the dry powder fermenter obtained is small in size and low in moisture content, which makes it easy to be transported and stored. Moreover, the dry powder fermenter prepared by this method has high fermentation performance, can rapidly acidify the food matrix, produce certain metabolites or inhibit or reduce the growth of spoilage/pathogenic flora by competitive exclusion mechanism, improving the shelf life and biosafety of products, in addition to its ability to promote the production of flavor of indigenous fermented meat products.

In the present disclosure, the number of lactic acid bacteria in multiplication culture and the survival rate after vacuum freeze-drying are determined by plate counting method, the cell membrane composition of lactic acid bacteria is analyzed by gas chromatography, the surface and internal morphology of the bacterial cells before and after freeze-drying are observed by scanning electron microscopy and transmission electron microscopy, and the metabolism enzyme activity of the bacterial cells is measured by using a kit, which mainly includes lactate dehydrogenase, pyruvate kinase, and ATPase, to assess the viability of the strain. Due to the formation of a protective layer of bacterial gelatin clusters between the bacterial cells in high concentration, the area exposed to unfavorable environments is reduced, and the addition of fatty acids changes the composition and formulation of the freeze-drying protective agent, thereby protecting the integrity of the cell membranes of the bacterial cells and maintaining the fluidity of the cell membranes. In this way, the prepared dry powder fermenter has excellent survival rate and high vitality, and the fermented meat products prepared by using this dry powder fermenter are of stable quality, and the flavor has been significantly improved with short fermentation time.

### Sequencing Results:

SEQ No. 1 Sequence of Lactiplantibacillus plantarum molecule YR07:
CAGGGTTATGGCTGACAGCTCGTACATAACGGGTCGAACAGAACTCTGGTATTGATTGGTGCTTGCATCAT
GATTTACATTTGAGTGAGTGGCGAACTGGTGAGTAACACGTGGGAAACCTGCCCAGAAGCGGGGGATAACACCTGGA
AACAGATGCTAATACCGCATAACAACTTGGACCGCATGGTCCGAGCTTGAAAGATGGCTTCGGCTATCACTTTTGGA
TGGTCCCGCGGCGTATTAGCTAGATGGTGGGGTAACGGCTCACCATGGCAATGATACGTAGCCGACCTGAGAGGGTA
ATCGGCCACATTGGGACTGAGACACGGCCCAAACTCCTACGGGAGGCAGCAGTAGGGAATCTTCCACAATGGACGAA
AGTCTGATGGAGCAACGCCGCGTGAGTGAAGAAGGGTTTCGGCTCGTAAAACTCTGTTGTTAAAGAAGAACATATCT
GAGAGTAACTGTTCAGGTATTGACGGTATTTAACCAGAAAGCCACGGCTAACTACGTGCCAGCAGCCGCGGTAATAC
GTAGGTGGCAAGCGTTGTCCGGATTTATTGGGCGTAAAGCGAGCGCAGGCGGTTTTTTAAGTCTGATGTGAAAGCCT
TCGGCTCAACCGAAGAAGTGCATCGGAAACTGGGAAACTTGAGTGCAGAAGAGGACAGTGGAACTCCATGTGTAGCG
GTGAAATGCGTAGATATATGGAAGAACACCAGTGGCGAAGGCGGCTGTCTGGTCTGTAACTGACGCTGAGGCTCGAA
AGTATGGGTAGCAAACAGGATTAGATACCCTGGTAGTCCATACCGTAAACGATGAATGCTAAGTGTTGGAGGGTTTC
CGCCCTTCAGTGCTGCAGCTAACGCATTAAGCATTCCGCCTGGGGAGTACGGCCGCAAGGCTGAAACTCAAAGGAAT
TGACGGGGGCCCGCACAAGCGGTGGAGCATGTGGTTTAATTCGAAGCTACGCGAAGAACCTTACCAGGTCTTGACAT
ACTATGCAAATCTAAGAGATTAGACGTTCCCTTCGGGGACATGGATACAGGTGGTGCATGGTTGTCGTCAGCTCGTG
TCGTGAGATGTTGGGTTAAGTCCCGCAACGAGCGCAACCCTTATTATCAGTTGCCAGCATTAAGTTGGGCACTCTGG
TGAGACTGCCGGTGACAAACCGGAGGAAGGTGGGGATGACGTCAAATCATCATGCCCCTTATGACCTGGGCTACACA
CGTGCTACAATGGATGGTACAACGAGTTGCGAACTCGCGAGAGTAAGCTAATCTCTTAAAGCCATTCTCAGTTCGGA
TTGTAGGCTGCAACTCGCCTACATGAAGTCGGAATCGCTAGTAATCGCGGATCAGCATGCCGCGGTGAATACGTTCC
CGGGCCTTGTACACACCGCCCGTCACACCATGAGAGTTTGTAACACCCAAAGTCGGTGGGGTAACCTTTGAGGAACC
AGCCTACCTCATCGAGTTTCCCCCG.
Latilactobacillus sakei L. 48
AGGGGGCGGGGTCTATACATGGCGGGGTCGAACGCACTCTCGTTTAGATTGAAGGAGCTTGCTCCTGATTG
ATAAACATTTGAGTGAGTGGCGGACGGGTGAGTAACACGTGGGTAACCTGCCCTAAAGTGGGGGATAACATTTGGAA
ACAGATGCTAATACCGCATAAAACCTAACACCGCATGGTGTAGGGTTGAAAGATGGTTTCGGCTATCACTTTAGGAT
GGACCCGCGGTGCATTAGTTAGTTGGTGAGGTAAAGGCTCACCAAGACCGTGATGCATAGCCGACCTGAGAGGGTAA
TCGGCCACACTGGGACTGAGACACGGCCCAGACTCCTACGGGAGGCAGCAGTAGGGAATCTTCCACAATGGACGAAA
GTCTGATGGAGCAACGCCGCGTGAGTGAAGAAGGTTTTCGGATCGTAAAACTCTGTTGTTGGAGAAGAATGTATCTG
ATAGTAACTGATCAGGTAGTGACGGTATCCAACCAGAAAGCCACGGCTAACTACGTGCCAGCAGCCGCGGTAATACG
TAGGTGGCAAGCGTTGTCCGGATTTATTGGGCGTAAAGCGAGCGCAGGCGGTTTCTTAAGTCTGATGTGAAAGCCTT
CGGCTCAACCGAAGAAGTGCATCGGAAACTGGGAAACTTGAGTGCAGAAGAGGACAGTGGAACTCCATGTGTAGCGG
TGAAATGCGTAGATATATGGAAGAACACCAGTGGCGAAGGCGGCTGTCTGGTCTGTAACTGACGCTGAGGCTCGAAA
GCATGGGTAGCAAACAGGATTAGATACCCTGGTAGTCCATGCCGTAAACGATGAGTGCTAGGTGTTGGAGGGTTTCC
GCCCTTCAGTGCCGCAGCTAACGCATTAAGCACTCCGCCTGGGGAGTACGACCGCAAGGTTGAAACTCAAAGGAATT
GACGGGGGCCCGCACAAGCGGTGGAGCATGTGGTTTAATTCGAAGCAACGCGAAGAACCTTACCAGGTCTTGACATC
CTTTGACCACTCTAGAGATAGAGCTTTCCCTTCGGGGACAAAGTGACAGGTGGTGCATGGTTGTCGTCAGCTCGTGT
CGTGAGATGTTGGGTTAAGTCCCGCAACGAGCGCAACCCTTATTACTAGTTGCCAGCATTTAGTTGGGCACTCTAGT
GAGACTGCCGGTGACAAACCGGAGGAAGGTGGGGACGACGTCAAATCATCATGCCCCTTATGACCTGGGCTACACAC
GTGCTACAATGGATGGTACAACGAGTTGCGAGACCGCGAGGTTTAGCTAATCTCTTAAAACCATTCTCAGTTCGGAT
TGTAGGCTGCAACTCGCCTACATGAAGCCGGAATCGCTAGTAATCGCGGATCAGCATGCCGCGGTGAATACGTTCCC
GGGCCTTGTACACACCGCCCGTCACACCATGAGAGTTTGTAACACCCAAAGCCGGTGAGGTAACCCTTCGTGGAGCT
CAGCTGTCTAAAGTTAAATCGGGATG.
Lactobacillus curvatus D2
CGGAACGCACTCTCGTTAGATTGAAGAAGCTTGCTTCTGATTGATAACATTTGAGTGAGTGGCGGACGGGT
GAGTAACACGTGGGTAACCTGCCCTAAAGTGGGGGATAACATTTGGAAACAGATGCTAATACCGCATAAAACCTAGC
ACCGCATGGTGCAAGGTTGAAAGATGGTTTCGGCTATCACTTTAGGATGGACCCGCGGTGCATTAGTTAGTTGGTGA
GGTAAAGGCTCACCAAGACCGTGATGCATAGCCGACCTGAGAGGGTAATCGGCCACACTGGGACTGAGACACGGCCC
AGACTCCTACGGGAGGCAGCAGTAGGGAATCTTCCACAATGGACGAAAGTCTGATGGAGCAACGCCGCGTGAGTGAA
GAAGGTTTTCGGATCGTAAAACTCTGTTGTTGGAGAAGAACGTATTTGATAGTAACTGATCAGGTAGTGACGGTATC
CAACCAGAAAGCCACGGCTAACTACGTGCCAGCAGCCGCGGTAATACGTAGGTGGCAAGCGTTGTCCGGATTTATTG
GGCGTAAAGCGAGCGCAGGCGGTTTCTTAAGTCTGATGTGAAAGCCTTCGGCTCAACCGAAGAAGTGCATCGGAAAC
TGGGAAACTTGAGTGCAGAAGAGGACAGTGGAACTCCATGTGTAGCGGTGAAATGCGTAGATATATGGAAGAACACC
AGTGGCGAAGGCGGCTGTCTGGTCTGTAACTGACGCTGAGGCTCGAAAGCATGGGTAGCAAACAGGATTAGATACCC
TGGTAGTCCATGCCGTAAACGATGAGTGCTAGGTGTTGGAGGGTTTCCGCCCTTCAGTGCCGCAGCTAACGCATTAA
GCACTCCGCCTGGGGAGTACGACCGCAAGGTTGAAACTCAAAGGAATTGACGGGGGCCCGCACAAGCGGTGGAGCAT
GTGGTTTAATTCGAAGCAACGCGAAGAACCTTACCAGGTCTTGACATCCTTTGACCACTCTAGAGATAGAGCTTTCC
CTTCGGGGACAAAGTGACAGGTGGTGCATGGTTGTCGTCAGCTCGTGTCGTGAGATGTTGGGTTAAGTCCCGCAACG
AGCGCAACCCTTATTACTAGTTGCCAGCATTTAGTTGGGCACTCTAGTGAGACTGCCGGTGACAAACCGGAGGAAGG
TGGGGACGACGTCAAATCATCATGCCCCTTATGACCTGGGCTACACACGTGCTACAATGGATGGTACAACGAGTCGC
GAGACCGCGAGGTTTAGCTAATCTCTTAAAACCATTCTCAGTTCGGATTGTAGGCTGCAACTCGCCTACATGAAGCC
GGAATCGCTAGTAATCGCGGATCAGCATGCCGCGGTGAATACGTTCCCGGGCCTTGTACACACCGCCCGTCACACCA
TGAGAGTTTGTAACACCCAAAGCCGGTGAGGTAACCTTCGGGAGCCAGCCGTCA.

The foregoing is intended only as an explanation of the embodiments of the present disclosure, and is not intended to limit the present disclosure in any way; therefore, any modification or alteration of the present disclosure made in the same spirit of the present disclosure should still be included in the scope of the present disclosure.

What is claimed is:

1. A dry powder fermenter, comprising Lactiplantibacillus plantarum YR07, Latilactobacillus sakei L. 48, and Lactobacillus curvatus D2; wherein the Lactiplantibacillus plantarum YR07 has been conserved in China Center for Type Culture Collection (CCTCC), with a conservation date of Aug. 18, 2022, and a strain conservation number of CCTCC NO:M 20221303; the Latilactobacillus sake]L. 48 has been conserved in the CCTCC, with a conservation date of Aug. 18, 2022, and a strain conservation number of CCTCC NO: M 20221306; the Lactobacillus curvatus D2 has been conserved in the CCTCC, with a conservation date of Feb. 21, 2023, and a strain conservation number of CCTCC NO: M 2023162.

2. A preparation method of the dry powder fermenter according to claim 1, comprising:
Step 1: inoculating the Lactiplantibacillus plantarum YR07, the Latilactobacillus sakei L. 48, and the Lactobacillus curvatus D2 into an MRS broth medium, respectively, and incubating at 35-39° C. at a rotational speed of 120-160 rpm under shaking; carrying out passaging culture for 3 times at an inoculum of 1.8-2.2%; adding strains to an MRS broth medium containing a multiplication factor at an inoculum of 1.8-2.2%, and culturing at 35-39° C. with a rotational speed of 120-160 rpm under shaking;
Step 2: when an optical density of a culture resulted in step 1 reaches 0.8-1.2, centrifuging and collecting the culture to obtain Lactiplantibacillus plantarum YR07 bacterial cells, Latilactobacillus sakei L. 48 bacterial cells, and Lactobacillus curvatus D2 bacterial cells; and
Step 3: suspending the Lactiplantbacillus plantarum YR07 bacterial cells, Latilactobacillus sakei L. 48 bacterial cells, and Lactobacillus curvatus D2 bacterial cells in sterile saline, mixing with a skimmed milk solution containing a freeze-drying protective agent and a fatty acid, and performing freeze-drying to obtain the dry powder fermenter.

3. The preparation method according to claim 2, wherein the multiplication factor is at least one of an amino acid, a buffer salt, and vegetable juice.

4. The preparation method according to claim 3, wherein the amino acid is at least one of aspartic acid, phenylalanine, histidine, valine, and lysine, at a concentration of 0.002 wt %-0.008 wt %.

5. The preparation method according to claim 2, wherein the buffer salt is: disodium hydrogen phosphate and sodium dihydrogen phosphate, disodium hydrogen phosphate and citric acid, potassium dihydrogen phosphate and sodium hydroxide, sodium acetate, potassium hydrogen phosphate and sodium citrate, or disodium hydrogen phosphate and potassium dihydrogen phosphate.

6. The preparation method according to claim 2, wherein the vegetable juice is at least one of tomato juice, carrot juice, and corn juice.

7. The preparation method according to claim 2, wherein in the step 1, a culture condition in each time of the passaging culture is the same: being cultured for 18-20 h at 35-39° C. at a rotational speed of 120-160 rpm; each liter of the MRS medium contains peptone 10.0 g, beef meal 5.0 g, yeast powder 4.0 g, glucose 20.0 g\Tween 80 1 mL\K₂HPO₄·7H₂O 2.0 g, triammonium citrate 2.0 g, magnesium sulfate 0.2 g; manganese sulfate, 0.05 g; pH 6.2±0.2;
in the step 2, a centrifugal force is 6000-10000 g; a centrifugal time is 10-30 min, and a temperature at centrifugation is 2-5° C.

8. The preparation method according to claim 2, wherein the freeze-drying protective agent is at least one of mannitol, sucrose, peptone, and alginate, and the fatty acid is at least one of oleic acid, linoleic acid, palmitic acid, and palmitoleic acid; a concentration of the fatty acid in the skimmed milk solution containing the freeze-drying protective agent and the fatty acid is 0.01 v/v %-0.05 v/v %; a concentration of the skimmed milk solution is 8 wt %-12 wt %; a volume ratio of a suspension of the bacterial cells to the skimmed milk solution containing the freeze-drying protective agent and the fatty acid is 2:1-1:2; and a duration of the freeze-drying is 24-30 h.

9. A preparation method of a cured meat product, using the dry powder fermenter according to claim 1.

10. The preparation method according to claim 9, wherein the cured meat product is a dry-cured duck, dry-cured chicken, traditional Chinese bacon (Dao-ban-xiang), or ham product.
